(19) United States
(12) Patent Application Publication
(10) Pub. No.: US 2024/0087751 A1
(43) Pub. Date: Mar. 14, 2024

(54) SYSTEMS AND METHODS FOR ORGAN SHAPE ANALYSIS FOR DISEASE DIAGNOSIS AND RISK ASSESSMENT

(71) Applicant: BrainScanology Inc., Cupertino, CA (US)

(72) Inventor: David H. Nguyen, Concord, CA (US)

(21) Appl. No.: 18/236,789

(22) Filed: Aug. 22, 2023

Related U.S. Application Data
(63) Continuation of application No. PCT/US2022/018961, filed on Mar. 4, 2022.
(60) Provisional application No. 63/157,576, filed on Mar. 5, 2021.

(51) Int. Cl. G16H 50/30; G06T 7/00; G16H 15/00

(52) U.S. Cl. CPC G16H 50/30; G06T 7/0012; G16H 15/00; G06T 2200/04; G06T 2207/10081; G06T 2207/10088; G06T 2207/10132; G06T 2207/20056; G06T 2207/20081; G06T 2207/20084; G06T 2207/30016; G06T 2207/30032

(57) ABSTRACT

A method for quantifying tissue morphology is provided. The method comprises receiving one or more images of a tissue, overlaying a grid system onto the one or more images, the grid system comprising a 180-degree radial system, 360-degree radial system, or a parallel lines system, and transforming the one or more images into compressed sums based on the grid system. The compressed sums may then be transformed into frequencies, for example using FFT. Machine learning may be used to provide a patient risk for developing a disease or indicate a diagnostic status of the tissue. In some embodiments, the tissue may be a brain.

### CROSS-REFERENCE

This application is a continuation application of International Patent Application No. PCT/US2022/018961, filed Mar. 4, 2022, which claims priority to U.S. Provisional Application No. 63/157,576, filed Mar. 5, 2021, each of which is entirely incorporated herein by reference.

### BACKGROUND

Many disease states are associated with abnormal structural and pathological tissue characteristics compared to healthy tissue. While histopathology and other imaging modalities can be used to distinguish between normal and abnormal tissues, quantifying spatially complex biological samples remains a challenge.

### SUMMARY

It would therefore be desirable to provide reliable, objective systems and methods for quantifying complex two-dimensional shapes. Additionally, it would be desirable to provide systems and methods for utilizing such quantitative information to classify images, such as medical images. Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

The present disclosure is directed to systems and methods which allow a user to objectively and precisely express complex shapes in quantitative form. The Linearized Compressed Polar Coordinates (LCPC) Transform in combination with a Fast Fourier Transform (FFT) can be used to objectively translate complex two-dimensional shapes into the frequencies, that describe those shapes. For example, ameboid shapes, such as those exhibited by folds of the brain, can be quantified using the systems and methods described herein. The information extracted by the LCPC Transform can then be used to train machine learning models in order to classify uncharacterized (e.g., patient) images into categories, such as: normal vs. diseased, low-risk vs. high-risk, young vs. old, etc.

In an aspect, a method for predicting a diagnostic status or a disease risk for a subject at risk of the disease, based at least in part from a two-dimensional (2D) image of tissue from the subject. The method comprises (a) overlaying a coordinate grid on the 2D image, wherein the coordinate grid comprises one or more lines producing one or more intersections with one or more features of the 2D image when overlaid thereon. The method also comprises (b) performing a linearized compressed polar coordinates (LCPC) transform based on the 2D image with the overlaid coordinate grid, wherein the LCPC transform comprises a sinusoidal representation of the one or more intersections. The method also comprises (c) performing at least one pre-processing operation on the LCPC transform. The method also comprises (d) determining the diagnostic status or the disease risk of the subject by using a trained algorithm to analyze the pre-processed LCPC transform. The method also comprises (e) providing the determined diagnostic status or the disease risk in an electronic report.

In some embodiments, the electronic report is provided to an operator of the subject.

In some embodiments, the operator is a medical professional.

In some embodiments, the electronic report is provided via an electronic display.

In some embodiments, the at least one pre-processing operation transforms the LCPC transform into a set of frequencies.

In some embodiments, the at least one pre-processing operation is a Fast Fourier Transform (FFT).

In some embodiments, performing the LCPC transform comprises (i) calculating one or more distances of intersections along a line of the one or more lines; (ii) along the line of the one or more lines, generating a sum of the one or more distances of intersections; and (iii) generating first and second LCPC coordinates, wherein a first coordinate is an index of the line of the one or more lines and a second coordinate is the sum of the one or more distances of intersections.

In some embodiments, a distance of the one or more distance is between an origin and a location of an intersection.

In some embodiments, the disease is one or more of bipolar disorder, schizophrenia, Alzheimer's disease, dementia, attention deficit hyperactivity disorder (ADHD), or autism.

In some embodiments, the 2D image is a medical image

In some embodiments, the 2D image is an MRI image.

In some embodiments, the 2D image is a CT image.

In some embodiments, the 2D image is an ultrasound image.

In some embodiments, the 2D image is a slice or cross-section of a 3D image of a tissue.

In some embodiments, the diagnostic status is a colon polyp morphology.

In some embodiments, the diagnostic status is used to grade the colon polyp.

In some embodiments, the coordinate grid is an [x, y] coordinate grid, a 180-degree radial grid, a system of parallel lines, a horizontal grid, or a diagonally-rotate grid.

In some embodiments, the outlining is performed using a neural network.

In some embodiments, the trained algorithm comprises a trained machine learning algorithm.

In some embodiments, the trained machine learning algorithm is an artificial neural network, a decision tree, a support vector machine, a regression, or a Bayesian network.

In some embodiments, a feature is a shape within the image.

In some embodiments, the shapes may be associated with individual cells, inner walls of tissues, or outer walls of tissues.

In some embodiments, the shape is associated with a region of the brain.

In some embodiments, the shape is a brain fold.

In some embodiments, the region is a hippocampus or temporal lobe.

In some embodiments, in (b), the sinusoidal representation is a discrete sinusoidal representation.

In a first aspect, a method for quantifying brain morphology is disclosed. The method comprises receiving one or more MRI images of a patient's brain; segmenting the one or more MRI images to generate one or more outlines; overlaying a grid system comprising a plurality of grid lines onto one or more outlines, wherein the grid system comprises a 360-degree radial system of grid lines, a 180-degree radial system of grid lines, or a parallel lines system of grid lines; determining one or more overlap locations between the plurality of grid lines and the one or more outlines; determining a magnitude for each overlap location of the one or more overlap locations, the magnitude being a distance between the overlap location and an origin of the overlapped grid line; summing the magnitudes for each overlap location on a same grid line to generate a compressed sum for each grid line; and transforming the compressed sums into frequencies.

In another aspect, a method for quantifying brain morphology is disclosed. The method comprises receiving one or more MRI images of a patient's brain; overlaying a grid system onto the one or more MRI images, wherein the grid system comprises a 360-degree radial system, or a parallel lines system; and transforming the one or more MRI images into compressed sums based on the grid system.

In some embodiments, the method may further comprise transforming the compressed sums into frequencies.

In another aspect, a system is disclosed. The system comprises a processor configured with instructions. The processor is configured to receive one or more two-dimensional images of a patient's brain, overlay a grid system onto the one or more two-dimensional images, transform the one or more two-dimensional images into compressed sums based on the grid system, transform the compressed sums into frequencies, and deliver one or more outputs based on the frequencies, wherein the one or more outputs provide a patient risk for developing a disease or indicate a diagnostic status of a tissue.

In another aspect, a computer-implemented method is provided. The computer-implemented method comprises receiving one or more two-dimensional images of a patient's brain; transforming the one or more two-dimensional images into one or more LCPC plots using a Linearized Compressed Polar Coordinates (LCPC) Transform; transforming the one or more LCPC plots into one or more frequency spectra; analyzing the one or more frequency spectra using one or more machine learning techniques; and delivering one or more outcomes based on the analysis of the one or more frequency spectra, wherein the one or more outputs provide a patient risk for developing a disease or indicate a diagnostic status of a tissue.

In some embodiments, the disease may be one or more of bipolar disorder, Alzheimer's disease, dementia, attention deficit hyperactivity disorder (ADHD), or autism.

In another aspect, a method for quantifying brain morphology is provided. The method comprises receiving one or more images of a patient's brain; overlaying a grid system onto the one or more images, wherein the grid system comprises a 360-degree radial system, a 180-degree radial system, or a parallel line system; determining border crossings of a plurality of lines of the grid system in the one or more images; applying a transform to a data set of the border crossings; and delivering one or more outcomes based on the transform, wherein the one or more outputs provide a patient risk for developing a disease or indicate a diagnostic status of a tissue.

In some embodiments, the disease may be one or more of bipolar disorder, Alzheimer's disease, dementia, attention deficit hyperactivity disorder (ADHD), or autism.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a 180-degree radial grid system overlaid on an MRI image of the brain, in accordance with embodiments.

FIG. 2 shows a 360-degree radial grid system overlaid on an MRI image of the brain, in accordance with embodiments.

FIG. 3 shows a vertical grid system overlaid on an MRI image of the brain, in accordance with embodiments.

FIG. 4 shows a horizontal grid system overlaid on an MRI image of the brain, in accordance with embodiments.

FIGS. 5A-5D show application of the LCPC Transform to quantify colon polyp morphology, in accordance with embodiments. FIG. 5A shows a histological image of a tubular adenoma with a 180-degree radial grid system overlaid thereon. FIG. 5B shows an uncompressed contour of the shape identified with the grid system of FIG. 5A. FIG. 5C shows the LCPC plot of FIG. 5B. FIG. 5D shows a FFT spectrum of the LCPC plot of the FIG. 5C.

FIGS. 5E-5G show application of the LCPC Transform to quantify brain fold morphology, in accordance with embodiments. FIG. 5E shows a sagittal MRI slice image of a brain. FIG. 5F shows an outline of the outer gray matter, as determined by pixel intensity thresholding, with a 180-degree radial grid system overlaid thereon. FIG. 5G shows a FFT spectrum of the LCPC plot.

FIGS. 6A-6F show application of the LCPC Transform to quantify brain morphology differences between normal and ADHD patients, in accordance with embodiments. FIGS. 6A and 6B show MRI images of normal and ADHD female brains, respectively, with 180-degree radial grid systems overlaid thereon. FIGS. 6C and 6D show average (n=3) FFT spectra of LCPC plots for normal and ADHD female brains, respectively. FIGS. 6E and 6F show an enlarged region of the spectra of FIGS. 6C and 6D, respectively.

FIG. 7 shows a flow chart of a method to apply the LCPC Transform to quantify and characterize an image, in accordance to embodiments.

FIG. 8 shows a flow chart of a method to train an artificial intelligence or machine learning model to characterize medical images using the LCPC Transform, in accordance with embodiments.

FIG. 9 shows a schematic diagram of a computer system programmed to run a machine learning model, in accordance with embodiments.

FIG. 10 illustrates a method for rotating two-dimensional (2D) slices of the brain with respect to axial, sagittal, and coronal views, to prepare the images for machine learning analysis, in accordance with embodiments.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present disclosure is described in relation to deployment of Linearized Compressed Polar Coordinates (LCPC) Transform systems and methods for brain fold analysis in MRI images. However, one of skill in the art will appreciate that this is not intended to be limiting and the devices and methods disclosed herein may be used in other anatomical areas and/or using other two-dimensional images. Anatomical areas may, for example, include the colon, the brain, bones (e.g., spinal vertebra, femoral head, etc.), the heart, the lungs, or the like. Images for analysis may, for example, include histological images, MRI images, 3D tomographic images, CT scans, or the like.

The embodiments as disclosed herein provide improved systems and methods for prediction of diagnostic status and/or disease risk in tissues. For example, application of the LCPC Transform to MRI brain scans may quantitatively describe shapes in brain folds, which may be used by a practitioner and/or by a computer system (e.g., for training machine learning models) to predict a diagnostic status and/or disease risk probability. Exemplary neurological diseases which may be assessed include bipolar disorder, Alzheimer's disease, dementia, schizophrenia, attention deficit hyperactivity disorder (ADHD), and autism. In another example, application of the LCPC Transform to colon histopathological images may quantitatively describe colon polyp morphology, which can then be used to grade the polyp (e.g., as normal colon, pedunculated adenoma, sessile serrated adenoma, etc.). Other diagnostic status and/or disease risk probabilities will be apparent to one of ordinary skill in the art based on the teachings herein.

The LCPC may provide human-interpretable “ground truth” data for machine-learned diagnostic predictions. Conventional deep learning analysis of anatomical or health-related images may make uninterpretable predictions based on features of the images which cannot be understood by health care professionals. As a result, these predictions may be of questionable value to the health care personnel. The LCPC, when applied to specific regions in the brain to produce numerical data for machine learning analysis, may inform the health care personnel exactly what part of the brain is being measured and may thus be interpretable to the health care personnel.

### LCPC Transform

The Linearized Compressed Polar Coordinates (LCPC) Transform can be used to objectively translate complex two-dimensional shapes into the frequencies, via the Fast Fourier Transform, that describe those shapes. Tissue architecture can be transformed into a complex sinusoid wave (i.e., LCPC plot) composed of discrete points. The LCPC plot can optionally undergo a Fast Fourier Transform (FFT) to obtain a spectrum of frequencies that represents the LCPC plot. The LCPC plot and/or the FFT spectrum thereof can serve as a signature of the spatial complexity (i.e., an index) within the imaged biological sample. The LCPC Transform can be used to detect subtle changes in architecture (e.g., tissue architecture), or cellular components, or biological samples (e.g., tissues), classify the complexity of tissue architecture as a simple collection of waves/frequencies, and/or identify and predict clinical status, outcomes, and/or response to therapies.

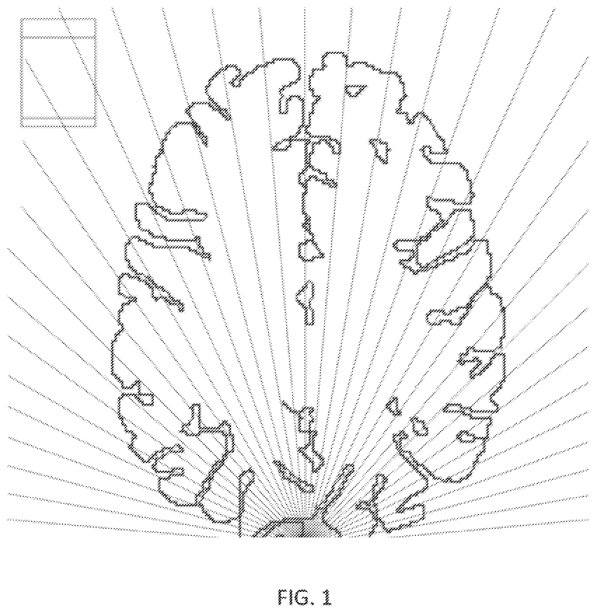

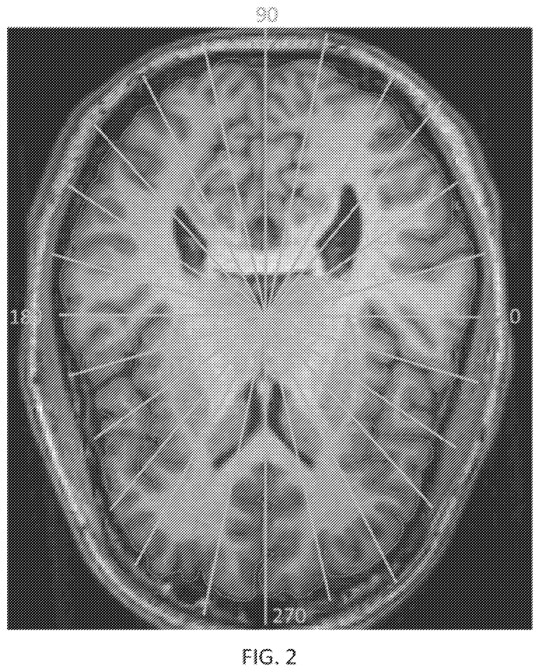

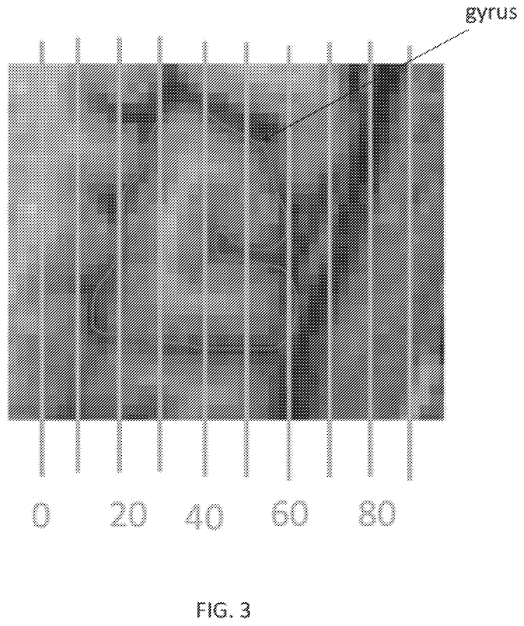

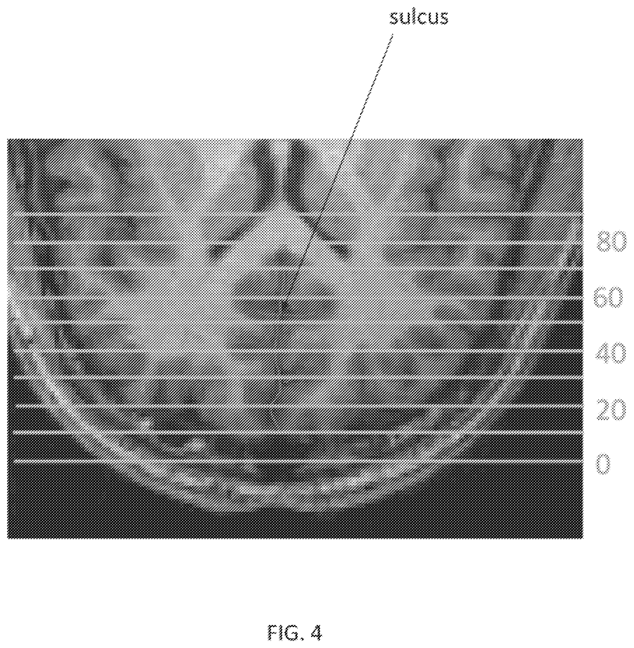

The LCPC Transform may overlay a grid system onto an image and a user (or a processor, if automated) can map two-dimensional (2D) coordinates (i.e., [x, y]) describing every location in which the lines of the grid system intersect the edges of any shape in the image. Shapes may include individual cells, inner walls of tissues (e.g., lumens), outer walls of tissue (e.g., organs, tubular structures, etc.), etc. Each intersection along a particular grid line may have a length described as the linear distance between the intersection and the baseline coordinate. Grid systems can have one origin as a baseline coordinate, in the case of a radial grid system (e.g., as shown in FIGS. 1 and 2), or many origins, such as the starting points of each line in a set of horizontal or vertical lines (e.g., as shown in FIGS. 3 and 4). Furthermore, the distances of each intersection along the same grid line can be summed into one value. Thus, each line in a grid system may have one corresponding summed distances value. This summing step may be referred to as “compressing”. This results in pairs of data that can be graphed in 2D: the x-coordinate may represent the numerical order of the lines in the grid system; the y-coordinate may represent the summed distance value associated with each line in the grid system. Plotting the compressed sums of each line versus the angle measure of each line (or other coordinate location of the line, as desired) may result in a 2D graph comprising a discrete sinusoid wave (also referred to herein as an LCPC plot). The LCPC plot, as with other sinusoidal data, may be amenable to the Fast Fourier Transform (FFT) that can turn the wave into a set of frequencies. Thus, each shape in the original image can be described as a distinct set of frequencies by using the LCPC Transform in combination with FFT. Since frequencies are precise quantities, the LCPC Transform may be highly sensitive in measuring shapes, particularly compared to the traditional approaches of measuring area, surface area, or volume.

The LCPC Transform may be used to capture spatial arrangement of cells, histological features, or other physiological/structural features identifiable in an image. Not only can the LCPC Transform capture the complexity of a tissue's outer contour, but it can be used to capture the spatial location of individual cells of interest (e.g., in immunohistochemically-stained images) within a tissue. The LCPC plot reduces the complexity of a tissue architecture into a small collection of wave frequencies. In doing so, the LCPC plot and/or FFT spectrum thereof, may be used to identify subsets of tissue structural and/or frequency profiles that represent non-obvious structural themes in tissues that correlate with different clinical outcome or biological behavior. Alternatively, or in combination, the LCPC plot and/or FFT spectrum thereof can be used to quantify differences in cellular/protein/chemical content between tissue types (e.g., inflammatory cell infiltration in normal vs. inflamed tissue) in tissue which may otherwise be gross-structurally similar to the naked eye. For example, the LCPC Transform, when used to detect the outer contour of a tissue along with a particular component therein (e.g., inflammatory cells) can quantitatively capture the subtly by subtracting one LCPC plot from another (e.g., an inflamed tissue plot minus the normal tissue).

The LCPC Transform may utilize a variety of grid systems. For example, the LCPC Transform may use a 180-degree radial grid as shown in FIG. 1, a 360-degree radial grid as shown in FIG. 2, or a system of parallel lines (e.g., a vertical grid as shown in FIG. 3, a horizontal grid as shown in FIG. 4, or a diagonally-rotate grid at specific angles). The nature of biological shapes, such as those in organs, tissues, and abnormal lesions, have physiological orientations (e.g. dorsal, ventral, rostral, caudal, medial, lateral). Capturing the spatial information in organs may involve user optimization as to what grid system is used and where it is placed on the image.

The grid systems used may have an order to them, for example, as represented by angle degrees. The number of segments defined by the grid system may be optimized to capture the complexity of a tissue architecture without creating undue work for the user/system. For example, two few segments may not adequately capture the complexity of tissue architecture while too many segments may unnecessarily create more work. Grid system(s) that best capture the biological information in a particular region of a complex organ may be designed and/or optimized to maximize how much spatial information is extracted from the shape, thereby reducing the noise in the output data and facilitating subsequent predictive machine learning models be more accurate for medical diagnostics.

In some embodiments, areas or shapes of interest may be outlined manually or automatically prior to performing the LCPC Transform. For example, automatic segmentation, otherwise known as outlining, can be done by thresholding the image based on pixel intensity or by neural networks. In some embodiments, automated scripts may be written (e.g., in the R programming language) for the purposes of segmentation and data extraction, as will be understood by one of ordinary skill in the art based on the teachings herein.

In some embodiments, the LCPC Transform may be applied to a single two-dimensional image in order to quantify a biological sample or tissue structure. In some embodiments, the LCPC Transform may be used to quantify biological samples or tissue structures in three dimensions (3D). For example, organs, such as brains, may generate many images during an MRI scan. Changes in brain folds due to age or disease may be viewed as 3D features. The LCPC Transform may be applied to consecutive 2D slices of an MRI of the brain and then, for each patient, the whole set of individual LCPC plots or frequency sets from the slices may be analyzed.

FIGS. 1-4 show exemplary grid systems which may be used for the LCPC Transform. FIG. 1 shows a 180-degree radial grid system overlaid on an MRI image of the brain. FIG. 2 shows a 360-degree radial grid system overlaid on an MRI image of the brain. FIG. 3 shows a vertical grid system overlaid on an MRI image of the brain. FIG. 4 shows a horizontal grid system overlaid on an MRI image of the brain. Areas or shapes of interest may be outlined manually or automatically prior to performing the LCPC Transform as described herein. For example, outer perimeter brain folds representing the outer edge of the gray matter may be outlined as shown in FIGS. 1 and 2, specific sections of the brain (e.g., a gyrus) may be outlined as shown in FIG. 3, or specific features of the brain (e.g., along the midline, a sulcus, etc.) may be traced as shown in FIG. 4.

Numerous published studies have reported that psychiatric diseases have detectable morphological changes in specific regions of the brain. For example, the hippocampus and temporal lobes of the brain can be affected in Alzheimer's disease. In bipolar disorder, the parietal lobe may be affected. These regions may be prime candidates for applying the LCPC Transform. For brain regions that span widest from the back of the brain to the front of the brain, such as the hippocampus, a grid of vertical lines that slice the hippocampus along the back-to-front axis may be more effective than a radial grid. Each grid system will extract spatial information, but which one gives the cleanest and more precise information may differ depending on the tissue of interest, feature of interest, and or image type/location, as will be understood by one of ordinary skill in the art. In some embodiments, the grid pattern may be optimized for use in machine learning models.

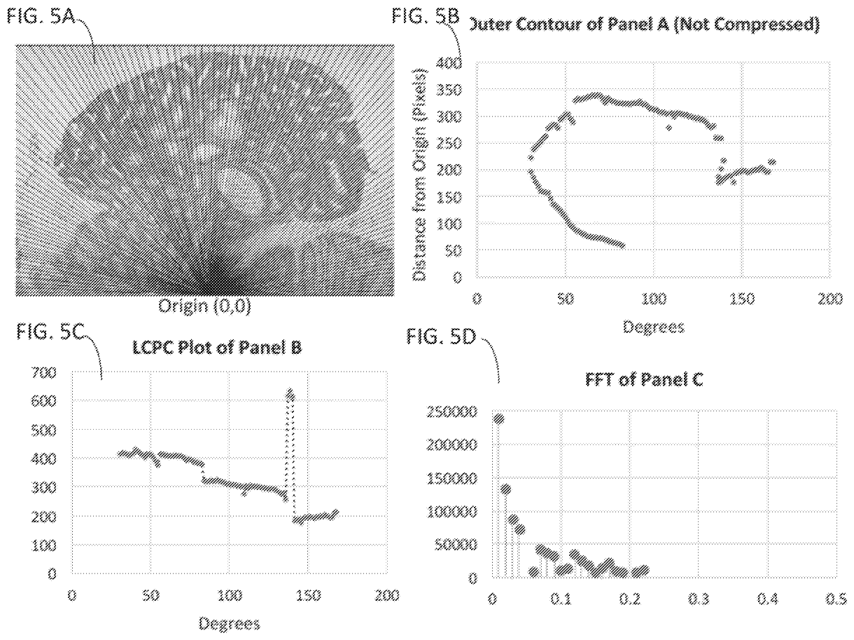

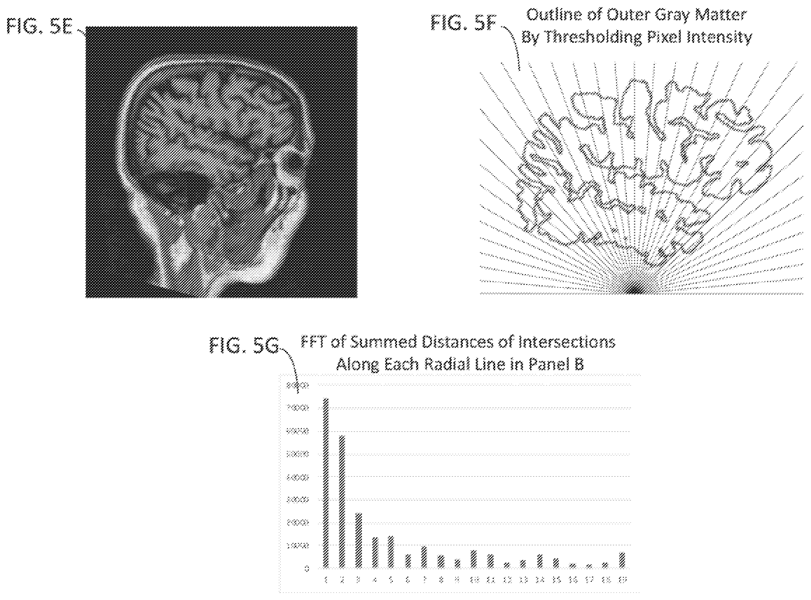

FIGS. 5A-5D show application of the LCPC Transform as described herein to quantify colon polyp morphology. FIG. 5A shows a histological image of a hematoxylin & eosin-stained tubular adenoma with a 180-degree radial grid system overlaid thereon. FIG. 5B shows an uncompressed contour of the shape identified with the grid system of FIG. 5A. Each of the grid lines of the grid system intersects features of the polyp, with each point of intersection being a vector having a magnitude (the distance from the vertex to the point). FIG. 5C shows the LCPC plot of FIG. 5B. The magnitudes of all points on the same grid line were summed to compress them into one numerical value representing the tissue architecture along its path. The compressed sums (y-axis) were plotted against the angle of the grid line on which they fall (x-axis). FIG. 5D shows a FFT spectrum of the LCPC plot of the FIG. 5C. The LCPC plot and/or FFT spectrum of a sample image may be compared, e.g., using machine learning techniques as described herein, to reference images/plots/spectra indicative of diagnostic status and/or disease.

FIGS. 5E-5G show application of the LCPC Transform to quantify brain fold morphology. FIG. 5E shows a sagittal MRI slice image of a brain. FIG. 5F shows an outline of the outer gray matter, as determined by pixel intensity thresholding, with a 180-degree radial grid system overlaid thereon. Each of the grid lines of the grid system intersecting the gray matter outline, with each point of intersection being a vector having a magnitude (the distance from the vertex to the point), were determined. The magnitudes of all points on the same grid line were summed to compress them into one numerical value representing the tissue architecture along its path. The compressed sums (y-axis) may optionally be plotted against the angle of the grid line on which they fall (x-axis) in an LCPC plot (not shown) as described herein. FIG. 5G shows a FFT spectrum of the LCPC plot. The LCPC plot and/or FFT spectrum of a sample image may be compared, e.g., using machine learning techniques as described herein, to reference images/plots/spectra indicative of diagnostic status and/or disease.

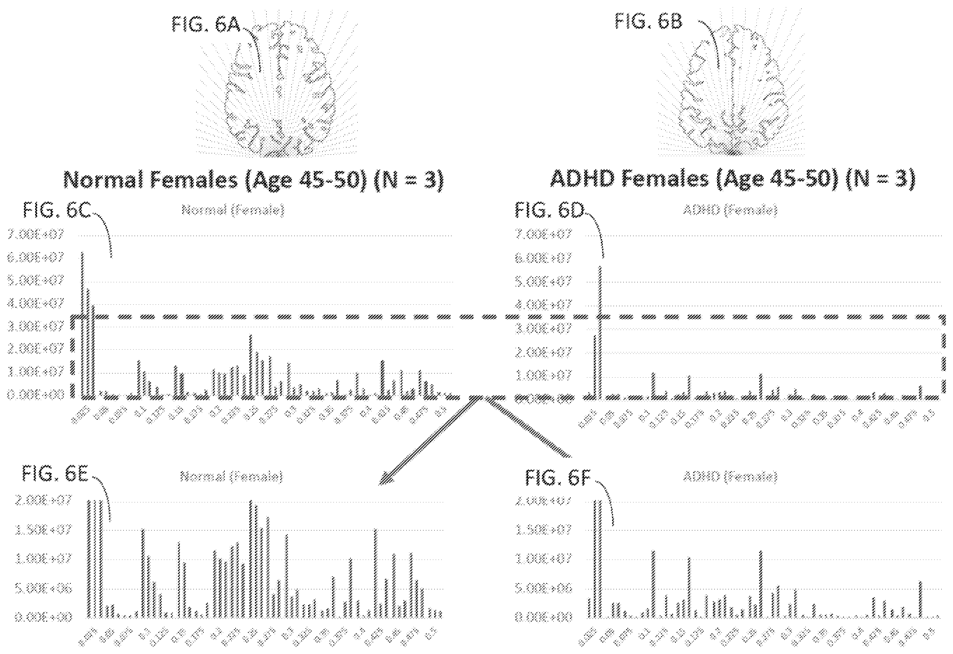

FIGS. 6A-6F show application of the LCPC Transform as described herein to quantify brain morphology differences between normal and ADHD patients. FIGS. 6A and 6B show MRI images (slice position is the first slice above corpus collosum) of normal and ADHD female brains, respectively, with 180-degree radial grid systems (with origins at the bottom edge of the occipital lobe) overlaid thereon. A 180-degree radial grid was selected for the LCPC Transform as an example. However, it will be understood that one of ordinary skill in the art that the LCPC Transform may be improved or optimized through various grid systems that also fit within the LCPC concept FIGS. 6C and 6D show average (n=3) FFT spectra of LCPC plots for normal and ADHD female brains, respectively. FIGS. 6E and 6F show an enlarged region of the spectra of FIGS. 66C and 6D, respectively, highlighting the distinct profiles of the normal and ADHD brain. The spectrograms in FIGS. 6E and 6F show clear differences in magnitude between the normal brain profiles and the ADHD brain profiles. Similar images may be used to create a robust machine learning model as described herein. Given enough samples, such distinct differences may be used to generate a machine learning model to determine a patient risk for developing ADHD using the teachings herein.

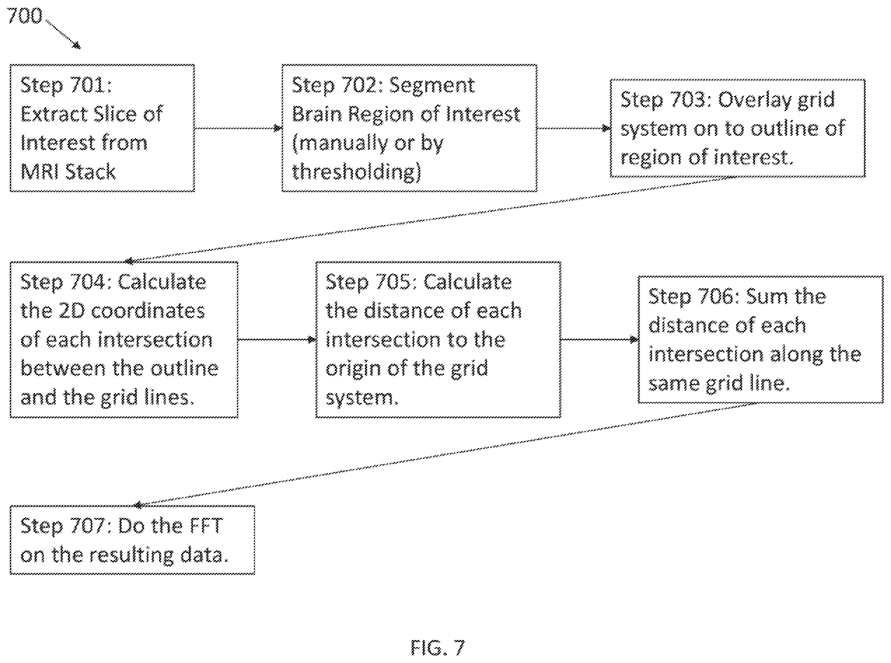

FIG. 7 shows a flow chart of a method 700 to apply the LCPC Transform to quantify and characterize an image.

At Step 701, an image of interest may be selected. The image of interest may, for example, be a slice from an MRI stack.

At Step 702, a region of interest within the image may be segmented, either manually or using automated image analysis techniques (e.g., thresholding).

At Step 703, a grid system may be overlaid onto the outline of the region of interest as described herein.

At Step 704, the 2D coordinates of each intersection between the outline and the gridlines may be calculated as described herein.

At Step 705, the distance of each intersection to the origin of the grid system may be calculated as described herein.

At Step 706, the distance of each intersection along the same grid line may be summed as described herein. Optionally, the compressed sums may be plotted as an LCPC plot as described herein.

At Step 707, an FFT spectrum of the data from Step 706 may be generated as described herein. The FFT spectrum and/or raw spectral data may be compared, e.g., using machine learning techniques as described herein, to reference images/plots/spectra indicative of diagnostic status and/or disease.

Although the steps above show a method of characterizing and quantifying an image in accordance with embodiments, a person of ordinary skill in the art will recognize many variations based on the disclosure provided herein. The steps may be completed in a different order. Steps may be added or deleted. Some of the steps may comprise sub-steps. Many of the steps may be repeated to achieve the desired characterization.

### Machine Learning

The methods and systems described herein may further utilize advanced computing techniques such as machine learning and/or neural networking in order to characterize the biological images and predict a diagnostic status, and/or disease risk probability.

Identification of quantitative structural differences between different diagnostic and/or diseased tissue states may be used to inform subsequent treatment decisions. As described herein, in a simplistic example, the degree, or lack thereof, of brain fold complexity may correlate with a diagnosis of ADHD. The LCPC Transform may provide a higher sensitivity for detecting ADHD-related differences in brain scans compared to traditional metrics (e.g., cortical volume, surface area, cortical thickness), and may enable earlier identification and treatment of the disease in patients. Alternatively, or in combination, the high sensitivity of the LCPC Transform may enable trained machine learning models to identify as-yet unknown subtypes that may have distinct clinical outcomes and treatment requirements.

Machine learning may leverage the multifactorial computing capability of a processor in order to assess the diagnostic status of a patient and/or determine the risk of developing disease for one or more input images (e.g., a single two-dimensional scan or a plurality of two-dimensional scans of a three-dimensional tissue). The machine learning system may be configured with instructions to analyze a large dataset of information (e.g., LCPC Transforms) with the potential to identify structural patterns of certain tissue types and correlate them to diagnostic status and/or disease state. The machine learning model may act as a database, storing individual patients' tissue characterization data. Alternatively, or in combination, the machine learning model may use a set of algorithms (e.g., instructions) to attempt to model high-level abstractions in the input data for example by using a deep graph with multiple processing layers to allow for a level of information about the relevant tissue/disease state not yet harnessed or understood with currently available methods.

The machine learning model may comprise an artificial neural network, a decision tree, a support vector machine, a regression analysis, a Bayesian network, logistic regression, or the like. For example, the machine learning model may comprise logistic regression or a support vector machine with a non-linear kernel trick.

The machine learning model may be trained using a training data set in which input data includes both the desired input images as well as known outcomes (e.g., diagnosis/disease state based on MRI scans, histological analysis, etc.). The system may “learn” from the exemplary training data set. The objective of the training process may be to approximate the function ƒ between the input and the output of a patient in order to later use the model to predict output values with high accuracy. The machine learning model may be trained on the raw spectra data (e.g., as shown in the FIGS. 5D, 5G, 6E, and 6F), or on numerical values that result from a normalization of the raw spectra data. Spectra data from non-affected individuals or affected individuals may serve as the ground truth categories on which the model may be trained. For example, MRI images from uncharacterized patients may have the exact same brain regions transformed by the LCPC Transform method and this data may be fed into the machine learning model for classification.

When there are enough examples in the system, the machine learning model can be used to predict the output value (e.g., diagnostic status) based on training with prior patient data. The model may be configured to report out binary outputs (e.g., “normal” or “diseased”). The model may be configured to report out non-binary outputs (e.g., “percent risk of disease”).

### Brain Alignment

Disclosed herein are methods for rotating and slicing 3D images of the brain so that the system may visualize regions of interest in 2D image slices derived from the 3D slices. These 2D slices may provide inputs for machine learning algorithms or statistical analysis used to predict disease risks or perform diagnostic tasks. For example, a 2D image with a region of interest prominently displayed may be analyzed by a convolutional neural network (CNN) to determine a disease risk. The exposed region of interest may improve the machine learning analysis, which otherwise may make a prediction on features that may be irrelevant to health care personnel.

The disclosed method may rotate a subject's brain with respect to x-, y-, and z-axes in a coordinate plane and produce the 2D slices of the brain at different orientations with respect to the coordinates. In some embodiments, the disclosed method may associate the axes of rotation with the axial, coronal, and sagittal views of the brain. However, other cross-sectional views of the brain may be used, depending on the condition the system is configured to detect. For example, a 3D image of the brain may be rotated incrementally (e.g., by five or fewer degrees per iteration) with respect to any one axis, any two axes, or all three coordinate axes. For example, each axis may be rotated from 0 to 360 degrees, such that there can be at least 361×361×361=47,045,881 triplet combinations by which to expose regions of interest affected by a disease status.

After each set of triplet rotations, the disclosed system may capture a 2D image slice of the 3D image. This measure of area of regions exposed by such a triplet rotation may be used for machine learning analysis prior to performing an LCPC transform, to quantify the shape of these regions. The triplet orientation system may reveal regions with area measures that may be used to train a machine learning model that may compete against machine learning models trained on LCPC outputs of the regions.

While a 3D image of the brain is rotated, a particular region of interest viewable in various 2D slices captured at various orientations may have a different area in any three of the slices selected. For example, the particular region may have a different area measure in each of the captured 2D slices. From analysis of measured areas of particular regions at different orientations, or analysis of the areas' shapes as quantified by the LCPC Transform at different orientations, the disclosed system may use machine learning to distinguish between healthy and diseased tissue.

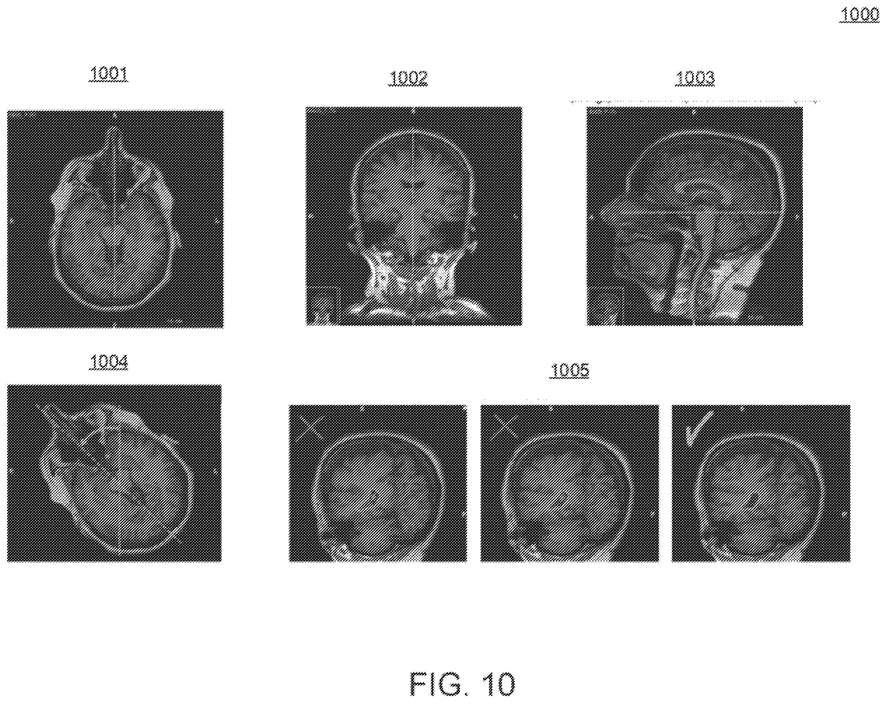

FIG. 10 illustrates a method for rotating two-dimensional (2D) slices of the brain with respect to axial, sagittal, and coronal views, to prepare the images for machine learning analysis, in accordance with embodiments.

In a first operation 1001, a second operation 1002, and third operation 1003, the system may calibrate by aligning, the axial, coronal, and sagittal views of the subject's brain along a first axis, a second axis, and third axis, respectively. This calibration may be performed to enable the system to analyze a subject's brain independent of skeletal or bone structure. Thus, the system may be able to analyze patients with vastly different physiologies or contexts. For example, a patient's scan may appear different from another's if the patient is injured or if the patient was in an odd position when the image was captured (e.g., lying on a bed in a prone or supine position). For example, as in operation 1003, the sagittal view may be aligned so that the dorsal apex of the pons is on the same horizontal axis as the dorsal apex of the cerebellum. The axial and coronal views may be then aligned along axes perpendicular to the cerebellum-pons axis. In other embodiments, the axial, coronal, and sagittal views may be aligned using a different set of anatomical features of the brain (e.g., the frontal lobe, hypothalamus, pituitary gland, temporal lobe, medulla oblongata, midbrain, thalamus, occipital lobe, or corpus callosum).

In a fourth operation 1004, the system may rotate the image. For example, the axial, coronal, and sagittal views of the brain may be rotated by 45 degrees.

In a fifth operation 1005, the system may select a particular view, following the rotation of operation, and determine one or more slices of the brain scan at that orientation that may contain a region of interest. For example, the system may collect slices of the sagittal view from anterior (e.g., the nose) to posterior (e.g., the back of the head) along the axis parallel to the sagittal profile (e.g., the cerebellum-pons axis). FIG. 10 illustrates that images may be captured until the region of interest is successfully located within one or more particular slices.

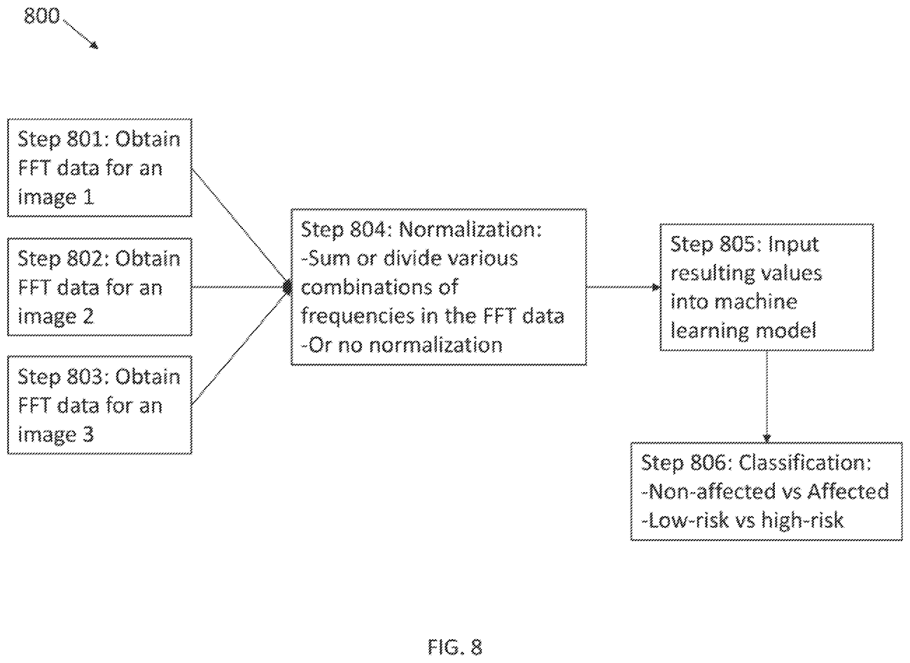

FIG. 8 shows a flow chart of a method 800 to train an artificial intelligence or machine learning model to characterize medical images using the LCPC Transform.

At Step 801, FFT data for a first image may be obtained as described herein.

At Step 802, FFT data for a second image may be obtained as described herein.

At Step 803, FFT data for a third image may be obtained as described herein.

Steps 801, 802, and/or 803 may be repeated as many times as necessary to obtain a sufficiently large sample set to train the machine learning model.

At Step 804, the FFT data obtained in Steps 801-803 may optionally be normalized. For example, various combinations of frequencies in the obtained FFT data may be summed or divided as will be understood by one of ordinary skill in the art. Alternatively, no normalization may be performed.

At Step 805, the FFT data (either raw data from Steps 801-803 or normalized data from Step 804) may be input into the machine learning model.

At Step 806, the machine learning model may classify the input FFT data. For example, the machine learning model may classify an input as non-affected vs. affected, low-risk vs. high-risk, etc. as described herein.

Although the steps above show a method of training an artificial intelligence or machine learning model to characterize medical images in accordance with embodiments, a person of ordinary skill in the art will recognize many variations based on the disclosure provided herein. The steps may be completed in a different order. Steps may be added or deleted. Some of the steps may comprise sub-steps. Many of the steps may be repeated to achieve the desired characterization or output.

Inputs to the machine learning model may include the raw spectra data resulting from the FFT, the numerical values resulting from a quantitative normalization the raw spectra data, and/or clinical data from the patients (age, gender, cognitive behavioral scores, etc.).

Outputs generated by the machine learning model may include age, gender, risk group (e.g., low, moderate, high), affected vs. non-affected, or the like as will be understood from one or ordinary skill in the art based on the teachings herein.

The outputs may be provided in an electronic report. The electronic report may include visualizations of the outputs, charts, graphs, or other representations suitable to provide an operator (e.g., a physician) with interpretable information usable for patient screening. The electronic report may be provided in a user interface (e.g., a graphical user interface (GUI) accessible by the operator.

### Example 1: Linear Regression Model for Bipolar Disorder

An exemplary machine learning model for diagnosing bipolar disorder males in their 20s or males in their 30s was trained on the spatial information extracted from 2D brain MRI images extracted by the LCPC Transform. 2D slices were isolated from the MRI stack and brain folds around the parietal region were segmented. An optimal grid system was chosen and overlaid on the segmented brain shapes. Locations of intersection between the grid and shape were extracted. These data were analyzed according to the LCPC method described herein and then underwent an FFT to produce a signature of frequencies representing patients with bipolar disorder or unaffected patients. These two sets of data were then used to train a linear regression model to receive similar input data from uncharacterized patient MRIs and output a predicted disease state.

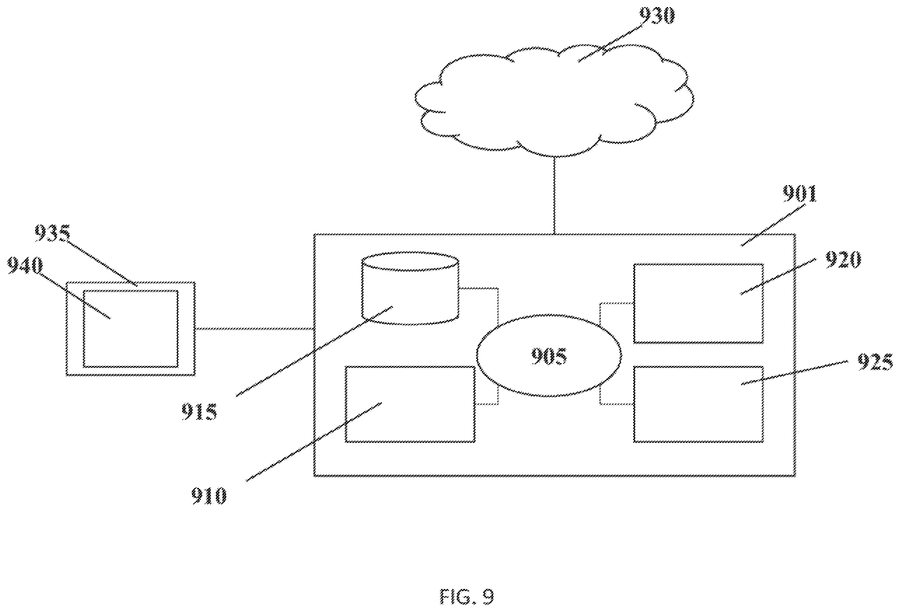

The present disclosure may provide computer control systems that can be programmed to implement methods of the disclosure. FIG. 9 shows a computer system 901 that can be programmed or otherwise configured to analyze data comprising tissue images and/or LCPC transforms of such images and determine diagnostic status and/or disease risk. The computer system 901 can regulate various aspects of any of the methods to determine the risk of disease and/or diagnostic status of the present disclosure, such as, for example, running the machine learning model described herein in order to take a set of relevant inputs from a user (and/or database) and transform them into an output such as a risk of a patient developing a disease as described herein. The computer system 901 can be an electronic device of a user or a computer system that is remotely located with respect to the electronic device. The electronic device can be a mobile electronic device.

The computer system 901 may include a central processing unit (CPU, also “processor” and “computer processor” herein) 905, which can be a single core or multi core processor, or a plurality of processors for parallel processing. The computer system 901 also may include memory or memory location 910 (e.g., random-access memory, read-only memory, flash memory), electronic storage unit 915 (e.g., hard disk), communication interface 920 (e.g., network adapter) for communicating with one or more other systems, and/or peripheral devices 925, such as cache, other memory, data storage and/or electronic display adapters. The memory 910, storage unit 915, interface 920 and/or peripheral devices 925 may be in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 3601 can communicate with a remote computer system of a user (e.g., a smart phone). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 3601 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing for example, patient sample input data or other input data to the machine learning model. Examples of UI's include, without limitation, a graphical user interface (GUI) and web-based user interface. Data input by a user into the user interface may be sent to the processor. The processor may be configured with instructions to run the machine learning model as described herein to generate one or more outputs. The output(s) of the machine learning model may be sent by the processor to a display which displays the outputs to a user with the user interface.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for predicting a diagnostic status or a disease risk for a subject at risk of the disease, based at least in part from a two-dimensional (2D) image of tissue from the subject, the method comprising:
(a) overlaying a coordinate grid on the 2D image, wherein the coordinate grid comprises one or more lines producing one or more intersections with one or more features of the 2D image when overlaid thereon;
(b) performing a linearized compressed polar coordinates (LCPC) transform based on the 2D image with the overlaid coordinate grid, wherein the LCPC transform comprises a sinusoidal representation of the one or more intersections;
(c) performing at least one pre-processing operation on the LCPC transform;
(d) determining the diagnostic status or the disease risk of the subject by using a trained algorithm to analyze the pre-processed LCPC transform; and
(e) providing the determined diagnostic status or the disease risk in an electronic report.

2. The method of claim 1, wherein the electronic report is provided to an operator of the subject.

3. The method of claim 2, wherein the operator is a medical professional.

4. (canceled)

5. The method of claim 1, wherein the at least one pre-processing operation transforms the LCPC transform into a set of frequencies.

6. The method of claim 1, wherein the at least one pre-processing operation is a Fast Fourier Transform (FFT).

7. The method of claim 1, wherein performing the LCPC transform comprises (i) calculating one or more distances of intersections along a line of the one or more lines; (ii) along the line of the one or more lines, generating a sum of the one or more distances of intersections; and (iii) generating first and second LCPC coordinates, wherein a first coordinate is an index of the line of the one or more lines and a second coordinate is the sum of the one or more distances of intersections.

8. The method of claim 7, wherein a distance of the one or more distances is between an origin and a location of an intersection.

9. The method of claim 1, wherein the disease is one or more of bipolar disorder, schizophrenia, Alzheimer's disease, dementia, attention deficit hyperactivity disorder (ADHD), or autism.

10. The method of claim 1, wherein the 2D image is a medical image.

11. The method of claim 10, wherein the 2D image is an MRI image, a CT image, or an ultrasound image.

12. (canceled)

13. (canceled)

14. The method of claim 10, wherein the 2D image is a slice or cross-section of a 3D image of a tissue.

15. The method of claim 1, wherein the diagnostic status is a colon polyp morphology.

16. The method of claim 15, wherein the diagnostic status is used to grade the colon polyp.

17. The method of claim 1, wherein the coordinate grid is an [x, y] coordinate grid, a 180-degree radial grid, a system of parallel lines, a horizontal grid, or a diagonally-rotate grid.

18. The method of claim 1, further comprising, prior to (b), outlining a shape of interest on the 2D image automatically or manually.

19. The method of claim 18, wherein the outlining is performed using a neural network.

20. The method of claim 1, wherein the trained algorithm comprises a trained machine learning algorithm.

21. The method of claim 20, wherein the trained machine learning algorithm is an artificial neural network, a decision tree, a support vector machine, a regression, or a Bayesian network.

22. The method of claim 1, wherein a feature is a shape within the image.

23. The method of claim 22, wherein the shape is associated with individual cells, inner walls of tissues, or outer walls of tissues.

24. The method of claim 23, wherein the shape is associated with a region of the brain.

25. The method of claim 24, wherein the shape is a brain fold.

26. The method of claim 24, wherein the region is a hippocampus or temporal lobe.

27. The method of claim 1, wherein, in (b), the sinusoidal representation is a discrete sinusoidal representation.
